(19) United States
(12) Patent Application Publication
(10) Pub. No.: US 2008/0112899 A1
(43) Pub. Date: May 15, 2008

(54) Carboxamides and Their Use

(76) Inventors: Christophe C. Galopin, Chesterfield, VA (US); Stefan Michael Furrer, Cincinnati, OH (US); Jay Patrick Slack, Loveland, OH (US); Pablo Victor Krawec, Cincinnati, OH (US); Lucienne Cole, Cincinnati, OH (US)

Correspondence Address: CURATOLO SIDOTI CO., LPA, 24500 CENTER RIDGE ROAD, SUITE 280, CLEVELAND, OH, 44145, US

(21) Appl. No.: 11/666,625

(22) PCT Filed: Nov. 21, 2005
(86) PCT No.: PCT/CH05/00687

§ 371 Date: May 25, 2007

(30) Foreign Application Priority Data
Nov. 23, 2004 (GB) 0425661.6

(51) Int. Cl. A61K 9/68; A61K 31/343; A61K 31/165; A61K 31/335

(52) U.S. Cl. 424/48; 514/462; 514/630; 564/196; 549/434

(57) ABSTRACT

The present invention refers to cooling compounds of formula I

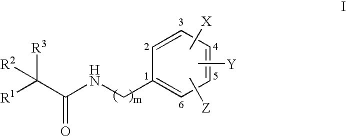

wherein R¹, R², R³, X, Y, Z, and m have the same meaning as given in the specification. The present invention refers furthermore to a process for their production and to product compositions comprising them.

The present invention refers to cooling compounds, namely compounds providing physiological cooling effects on the skin and on the mucous membranes of the mouth. The present invention refers furthermore to a process for their production and to product compositions comprising them.

In the flavor and fragrance industry there is an ongoing demand for compounds having unique cooling properties that provides the user with a pleasing cooling effect and which are suitable for use in a variety of products, particularly in ingestible and topical products.

British Patent GB 1,421,744 reports the discovery of simple N-substituted amides having a physiological cooling effect. These chemicals are versatile because they can be made completely synthetically. Their starting material does not rely on a natural source, in contrast with N-substituted p-menthanecarboxamides described in U.S. Pat. No. 4,150,052.

It has now been found that a certain class of carboxamides exhibits a strong cooling effect. Accordingly the invention refers in one of its aspects to the use of a compound of formula I as cooling agent

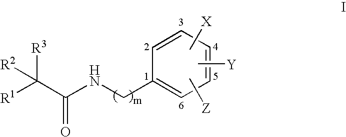

wherein X is (CH₂)ₙ—R, wherein R is a group comprising at least one free electron pair;

n is 0 or 1;

Y and Z are independently selected from H, OH, C1 to C4 alkyl, C1 to C4 alkoxy; or

Z is H, OH, C1 to C4 alkyl, or C1 to C4 alkoxy; and

X and Y form together a bivalent radical selected from the group consisting of —O—CH₂—O—, —N═CH—O—, —N—CH—NH—, and —N═CH—S— which forms together with the carbon atoms to which they are attached a 5-membered ring, i.e. a 1,3-dioxalane ring, a 1,3-oxazole ring, a 1,3-diazole ring or a 1,3-thiazole ring respectively;

m is 0 or 1;

R¹ is H, C1 to C4 alkyl, preferably H or methyl;

R² and R³ represent independently C1 to C4 alkyl, preferably branched C3 or C4 alkyl; and the sum of carbon atoms R¹+R²+R³ is at least 6.

Groups comprising at least one free electron pair are preferably selected from halogens, e.g. Cl, F, and Br, cyano, hydroxy, methoxy, NO₂, acetyl, SO₂NH₂, CHO, COOH, C1 to C4 alkyl carboxylate such as COOCH₃ and COOC₂H₅, C1 to C4 alkyl carboxamide such as CONHCH₃, and 5-membered heterocyclic rings comprising two or more hetero atoms selected from the group consisting of N, S, and O, such as diazole, triazole, tetrazole, oxazole and thiazole.

Compounds of formula I wherein R¹ is hydrogen and R² and R³ are iso-propyl or compounds wherein R¹ is methyl and R² and R³ are iso-propyl are particularly preferred.

Preferred compounds of formula I are also those wherein X is in 2, 4 or 6-position, i.e. in ortho or para. The most preferred compounds are when X is in 2, 4 or 6-position and Y and Z independently represents hydrogen, hydroxy, methoxy or methyl.

Surprisingly the inventors found that certain compounds of the present invention exhibit even stronger cooling effects than WS 23 (N,2,3-trimethyl-2-isopropylbutanamide) which can be considered as chemically distantly related to the compounds of the present invention. According to our best knowledge, WS 23 is the only compound disclosed in GB 1,421,744, which has been commercialised and has therefore been chosen as comparison compound. Thus, most preferred are compounds of formula I wherein m is 0, n is 0 and X is selected from the group consisting of cyano, methoxy, and methyl carboxylate (COOCH₃). Also preferred are compounds of formula I wherein m and n is 0 and X and Y taken together are O—CH₂—O, i.e. X and Y form together with the carbon atoms to which they are attached a dioxol ring.

The compounds of the present invention have never been described in literature and thus the present invention refers in a further aspect to a compound of formula I

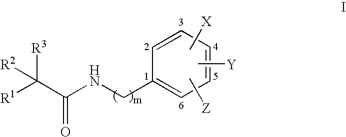

wherein R1, R2, R3, m, X, Y and Z has the same meaning as given above.

Particularly preferred compounds of formula I are N-(4-cyanophenyl) 2-isopropylisovaleramide, N-(4-cyanophenyl) 2-methyl-2-isopropylisovaleramide, N-(4-methoxyphenyl) 2-methyl-2-isopropylisovaleramide, N-(4-cyanomethyl-phenyl)-2-methyl-2-isopropyl-isovaleramide, 4-(2-isopropyl-2,3-dimethyl-butyrylamino)-benzoic acid isopropyl ester, N-(4-methoxyphenyl) 2-isopropyl-isovaleramide, N-(2-cyanophenyl) 2-isopropylisovaleramide, N-vanillyl 2-methyl-2-isopropylisovaleramide, and N-benzo[1,3]dioxol-5-yl 2-methyl-2-isopropylisovaleramide and.

Examples of other compounds falling within the scope of the present invention are N-vanillyl 2-isopropyl-isovaleramide and N-benzo[1,3]dioxol-5-yl 2-isopropylisovaleramide.

The compounds of the present invention may be used in products that are applied to the mouth or the skin to give a cooling sensation. By “applying” is meant any form of bringing into contact, for example, oral ingestion or, in the case of tobacco products, inhalation. In the case of application to the skin, it may be, for example, by including the compound in a cream or salve, or in a sprayable composition. The invention therefore also provides a method of providing a cooling effect to the mouth or skin by applying thereto a product comprising a compound as hereinabove described.

Products that are applied to the mouth may include foodstuffs and beverages taken into the mouth and swallowed, and products taken for reasons other than their nutritional value, e.g. tablets, mouthwash, throat sprays, dentifrices and chewing gum. Products that are applied to the skin may be selected from perfumes, toiletries, lotions, oils and ointment applicable to the skin of the human body, whether for medical or other reasons. Accordingly, the present invention refers in a further aspect to a composition comprising an amount of a compound of formula I, or a mixture thereof, sufficient to stimulate the cold receptors in the areas of the skin or mucous membrane with which the composition comes into contact and thereby promote the desired cooling effect. A cooling effect may be achieved upon application of a liquid product to the mucous membrane, e.g. mouth mucous membrane, comprising less than 5000 ppm, preferably between 300 and 3000 ppm, of a compound of formula I.

Thus the present invention further relates to an end-product selected from the group consisting of topical products, oral care products, nasal care products, toilet articles, ingestible products and chewing gum, which comprises a product base and an effective amount of a cooling compound of formula I, or a mixture thereof.

The compounds of the invention may be used alone or in combination with other cooling compounds known in the art, e.g. menthol, menthone, isopulegol, N-ethyl p-menthanecarboxamide (WS-3), N,2,3-trimethyl-2-isopropylbutanamide (WS-23), menthyl lactate, mono-menthyl succinate (PhyScool™), mono-menthyl glutarate, O-menthyl glycerine (CoolAct™ 10) and 2-sec-butylcyclohexanone (Freskomenthe™).

The compounds of formula I may be prepared by chlorination of an acid of the general formula R¹R²R³C—COOH to the corresponding acid chloride which is further reacted with an amine of formula II

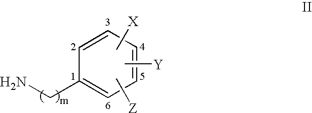

wherein R¹, R², and R³, m, X, Y and Z have the same meaning as given for the compounds of formula I under process conditions well known in the art. Certain acids of the formula R¹R²R³C—COOH are commercially available. In general they may be prepared for example by a method described in Tetrahedron, 1980, 36(6), 775-7 or Journal of Chemical Research, 1978, 2, 46.

The invention is now further described by means of the following non-limiting examples.

### EXAMPLE 1

### N-(4-cyanophenyl) 2-isopropylisovaleramide

To a flask were added 5.9 g (50 mmol) of 4-aminobenzonitrile, 4 mL of pyridine and 100 mL MtBE. To this mixture, 8 g of 2-isopropylisovaleryl chloride were added dropwise over 5 minutes. The reaction mixture was stirred for 24 h. To the reaction mixture, 50 mL of water were added. The mixture was separated. The organic layer was washed with 50 mL of water and 50 mL of brine. The organic layer was dried over MgSO₄. The solvent was evaporated in vacuo to afford the crude product, which was recrystallized from hexanes to afford 10 g of the desired product.

MS: 244([M⁺]), 229, 99, 57

¹H NMR (300 MHz; CDCl₃) δ: 7.68 (d, 2H), 7.61 (d, 2H), 7.2 (s, 1H), 2.11 (m, 2H), 1.77 (t, 1H), 1.01 (d, 6H), 0.99 (d, 6H).

### EXAMPLE 2

Following the same procedure according to Example 1 the compounds listed in Table 1 have been synthesised.

TABLE 1

| No. | Structure | Name | physical data |
|---|---|---|---|
| A | 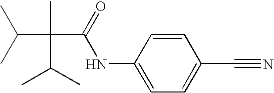 | N-(4-Cyanophenyl)2-methyl-2-isopropylisovaleramide | MS: 244([M⁺]), 229, 99, 57;¹H NMR (300 MHz; DMSO) δ: 7.67 (d, 2H)7.61 (d, 2H), 7.4 (s, 1H), 2.11 (m, 2H),1.16 (s, 3H), 0.98 (d, 6H), 0.93 (d, 6H).Melting point: 142° C. |
| B | 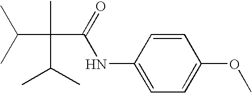 | N-(4-Methoxyphenyl)2-methyl-2-isopropylisovaleramide | MS: 263, 123, 108, 113, 57, 43 |
| C | 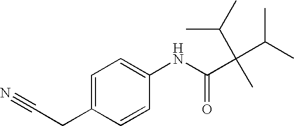 | N-(4-Cyanomethyl-phenyl)-2-methyl-2-isopropyl-isovaleramide | MS: 272, 132, 113, 57 |
| D | 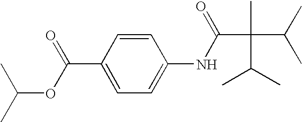 | 4-(2-Isopropyl-2,3-dimethyl-butyrylamino)-benzoic acidmethyl ester | MS: 291, 249, 151, 113, 57 |
| E | 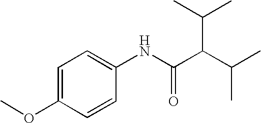 | N-(4-Methoxyphenyl)2-isopropylisovaleramide | MS: 249. 123, 108, 57, 49 |
| F | 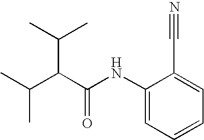 | N-(2-Cyanophenyl)2-isopropylisovaleramide | MS: 244, 229, 99, 57 |
| G | 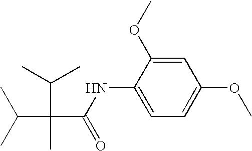 | N-(2,4-Dimethoxyphenyl)2-ethyl-2-isopropylisovaleramide | MS: 293, 278, 153, 138, 113, 57, 43 |
| H | 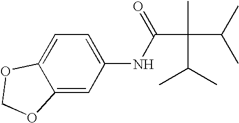 | N-Benzo[1,3]dioxol-5-yl 2-methyl-2-isopropylisovaleramide | MS: 277, 137, 113, 57, 43 |
| I | 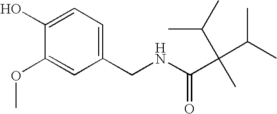 | N-Vanillyl 2-methyl-2-isopropylisovaleramide | MS: 293, 278, 250, 137, 57, 43 |

### EXAMPLE 3

### Cooling Effect

The cooling intensity of the compounds was determined by a trained panel of 4 to 8 people according to the isointensity method as described below.

Aqueous solutions of various concentrations of a chemical compound are prepared and tasted. The cooling intensity of each solution was compared to that of an aqueous solution of the reference compound at 2 ppm, namely N,2,3-trimethyl-2-isopropylbutanamde (WS 23). The results are given in the list below.

| Example | Chemical name | rel. cooling intensity |
|---|---|---|
| Ex. 1 | N-(4-Cyanophenyl) 2-isopropylisovaleramide | 1.3 |
| Ex. 2A | N-(4-Cyanophenyl) 2-methyl-2-isopropylisovaleramide | 1.5 |
| Ex. 2B | N-(4-Methoxyphenyl) 2-methyl-2-isopropylisovaleramide | 1.1 |
| Ex. 2C | N-(4-Cyanomethyl-phenyl)-2-methyl-2-isopropyl-isovaleramide | 0.2 |
| Ex. 2D | 4-(2-Isopropyl-2,3-dimethyl-butyrylamino)-benzoic acid methyl ester | 1.4 |
| Ex. 2E | N-(4-Methoxyphenyl) 2-isopropylisovaleramide | 1.1 |
| Ex. 2F | N-(2-Cyanophenyl) 2-isopropylisovaleramide | 1.7 |
| Ex. 2G | N-(2,4-Dimethoxyphenyl) 2-methyl-2-isopropylisovaleramide | 0.9 |
| Ex. 2H | N-Benzo[1,3]dioxol-5-yl 2-methyl-2-isopropylisovaleramide | 0.9 |
| Ex. 2I | N-Vanillyl 2-methyl-2-isopropylisovaleramide | 0.8 |

Example 4: Application in mouthwash

| | | |
|---|---|---|
| Alcohol 95% | 177 | mL |
| Sorbitol 70% | 250 | g |
| N-(4-Cyanomethylphenyl) 2-isopropylisovaleramide | 50 | mL |
| (1% sol. in alcohol) | | |
| Peppermint oil, Terpeneless | 0.300 | g |
| Methyl salicylate | 0.640 | g |
| Eucalyptol | 0.922 | g |
| Thymol | 0.639 | g |
| Benzoic acid | 1.500 | g |
| Pluronic ® F127 | 5.000 | g |
| Sodium Saccharin | 0.600 | g |
| Sodium Citrate | 0.300 | g |
| Citric Acid | 0.100 | g |
| Water | q.s. 1 | liter |

Pluronic ® F127 is a difunctional block copolymer surfactant (Trade mark of BASF)

All the ingredients are mixed. 30 mL of obtained solution is put in the mouth, swished around, gargled and spit out. A strong cooling sensation is felt in every area of the mouth as well as lips.

Example 5: Application in toothpaste

| | |
|---|---|
| Basic opaque toothgel without flavor or fragrance | 97.000 g |
| N-(4-cyanophenyl) 2-methyl-2-isopropylisovaleramide | 2.500 g |
| (2% sol. in PG*) | |
| Peppermint oil, Terpeneless | 0.500 g |

*PG = Propylen glycole

The chemicals are mixed in the toothgel, and 1 g of the toothgel is put on a toothbrush and a panelist's teeth are brushed. The mouth is rinsed with water and the water is spit out. A strong, icy cooling sensation is felt by the panelist in all areas of the mouth.

1. A compound of formula I 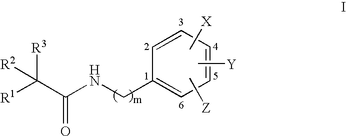
wherein X is (CH₂)ₙ—R, wherein R is a group comprising at least one free electron pair and n is 0 or 1;
Y and Z are independently selected from H, OH, C1 to C4 alkyl, C1 to C4 alkoxy; or
Z is H, OH, C1 to C4 alkyl, or C1 to C4 alkoxy; and
X and Y form together a bivalent radical selected from the group consisting of —O—CH₂—O—, —N═CH—O—, —N═CH—NH—, and —N═CH—S— which forms together with the carbon atoms to which they are attached a 5-membered ring;
m is 0 or 1;
R¹ is H, C1 to C4 alkyl;
R² and R³ represent independently C1 to C4 alkyl; and
the sum of carbon atoms R¹+R²+R³ is at least 6.

2. A compound according to claim 1 wherein R² and R³ represent independently a branched C3 or C4 alkyl.

3. A compound according to claim 1 wherein R¹ is H or methyl and R² is iso-propyl and R³ is iso-propyl.

4. A compound according to claim 1 wherein R is selected from the group consisting of Cl, F, Br, cyano, hydroxyl, methoxy, NO₂, acetyl, SO₂NH₂, CHO, COOH, C1 to C4 alkyl carboxylate, C1 to C4 alkyl carboxamide, and 5-membered heterocyclic rings comprising two or more hetero atoms selected from the group consisting of N, S, and O.

5. A compound according to claim 1 selected from the group consisting of compounds of formula I are N-(4-cyanophenyl) 2-isopropylisovaleramide, N-(4-cyanophenyl) 2-methyl-2-isopropylisovaleramide, N-(4-methoxyphenyl) 2-methyl-2-isopropylisovaleramide, N-(4-cyanomethyl-phenyl)-2-methyl-2-isopropyl-isovaleramide, 4-(2-isopropyl-2,3-dimethyl-butyrylamino)-benzoic acid isopropyl ester, N-(4-methoxyphenyl) 2-isopropyl-isovaleramide, N-(2-cyanophenyl) 2-isopropylisovaleramide, N-vanillyl 2-methyl-2-isopropylisovaleramide, N-vanillyl 2-isopropyl-isovaleramide, N-benzo[1,3]dioxol-5-yl 2-methyl-2-isopropylisovaleramide and N-benzo[1,3]dioxol-5-yl 2-isopropylisovaleramide.

6. (canceled)

7. A method of providing a cooling effect to the mouth or skin by applying thereto a product comprising a compound of formula I as defined in claim 1, or mixtures thereof.

8. A product that provides a cooling effect to the mouth or skin, which product comprises at least one compound selected from the group of compounds of formula I as defined in claim 1.

9. A product selected from the group consisting of topical products, oral care products, nasal care products, toilet articles, ingestible products and chewing gum, comprising a product base and an effective amount of a cooling compound of formula I as defined in claim 1, or a mixture thereof.

10. The product according to claim 9 wherein R² and R³ represent independently a branched C3 or C4 alkyl, optionally wherein R¹ is H or methyl and R² is iso-propyl and R³ is iso-propyl.

11. The product according to claim 9 wherein R is selected from the group consisting of Cl, F, Br, cyano, hydroxyl, methoxy, NO₂, acetyl, SO₂NH₂, CHO, COOH, C1 to C4 alkyl carboxylate, C1 to C4 alkyl carboxamide, and 5-membered heterocyclic rings comprising two or more hetero atoms selected from the group consisting of N, S, and O.

12. The product according to claim 9 wherein the compound of formula I is selected from the group consisting of compounds of formula I are N-(4-cyanophenyl) 2-isopropylisovaleramide, N-(4-cyanophenyl) 2-methyl-2-isopropylisovaleramide, N-(4-methoxyphenyl) 2-methyl-2-isopropylisovaleramide, N-(4-cyanomethyl-phenyl)-2-methyl-2-isopropyl-isovaleramide, 4-(2-isopropyl-2,3-dimethyl-butyrylamino)-benzoic acid isopropyl ester, N-(4-methoxyphenyl) 2-isopropyl-isovaleramide, N-(2-cyanophenyl) 2-isopropylisovaleramide, N-vanillyl 2-methyl-2-isopropylisovaleramide, N-vanillyl 2-isopropyl-isovaleramide, N-benzo[1,3]dioxol-5-yl 2-methyl-2-isopropylisovaleramide and N-benzo[1,3]dioxol-5-yl 2-isopropylisovaleramide.

13. The product according to claim 8 wherein R² and R³ represent independently a branched C3 or C4 alkyl, optionally wherein R¹ is H or methyl and R² is iso-propyl and R³ is iso-propyl.

14. The product according to claim 8 wherein R is selected from the group consisting of Cl, F, Br, cyano, hydroxyl, methoxy, NO₂, acetyl, SO₂NH₂, CHO, COOH, C1 to C4 alkyl carboxylate, C1 to C4 alkyl carboxamide, and 5-membered heterocyclic rings comprising two or more hetero atoms selected from the group consisting of N, S, and O.

15. The product according to claim 8 wherein the compound of formula I is selected from the group consisting of compounds of formula I are N-(4-cyanophenyl) 2-isopropylisovaleramide, N-(4-cyanophenyl) 2-methyl-2-isopropylisovaleramide, N-(4-methoxyphenyl) 2-methyl-2-isopropylisovaleramide, N-(4-cyanomethyl-phenyl)-2-methyl-2-isopropyl-isovaleramide, 4-(2-isopropyl-2,3-dimethyl-butyrylamino)-benzoic acid isopropyl ester, N-(4-methoxyphenyl) 2-isopropyl-isovaleramide, N-(2-cyanophenyl) 2-isopropylisovaleramide, N-vanillyl 2-methyl-2-isopropylisovaleramide, N-vanillyl 2-isopropyl-isovaleramide, N-benzo[1,3]dioxol-5-yl 2-methyl-2-isopropylisovaleramide and N-benzo[1,3]dioxol-5-yl 2-isopropylisovaleramide.

16. The method according to claim 7 wherein R² and R³ represent independently a branched C3 or C4 alkyl, optionally wherein R¹ is H or methyl and R² is iso-propyl and R³ is iso-propyl.

17. The method according to claim 7 wherein R is selected from the group consisting of Cl, F, Br, cyano, hydroxyl, methoxy, NO₂, acetyl, SO₂NH₂, CHO, COOH, C1 to C4 alkyl carboxylate, C1 to C4 alkyl carboxamide, and 5-membered heterocyclic rings comprising two or more hetero atoms selected from the group consisting of N, S, and O.

18. The method according to claim 7 wherein the compound of formula I is selected from the group consisting of compounds of formula I are N-(4-cyanophenyl) 2-isopropylisovaleramide, N-(4-cyanophenyl) 2-methyl-2-isopropylisovaleramide, N-(4-methoxyphenyl) 2-methyl-2-isopropylisovaleramide, N-(4-cyanomethyl-phenyl)-2-methyl-2-isopropyl-isovaleramide, 4-(2-isopropyl-2,3-dimethyl-butyrylamino)-benzoic acid isopropyl ester, N-(4-methoxyphenyl) 2-isopropyl-isovaleramide, N-(2-cyanophenyl) 2-isopropylisovaleramide, N-vanillyl 2-methyl-2-isopropylisovaleramide, N-vanillyl 2-isopropyl-isovaleramide, N-benzo[1,3]dioxol-5-yl 2-methyl-2-isopropylisovaleramide and N-benzo[1,3]dioxol-5-yl 2-isopropylisovaleramide.

19. The compound according to claim 4, wherein R² and R³ represent independently a branched C3 or C4 alkyl.

20. The compound according to claim 4 wherein R¹ is H or methyl and R² is iso-propyl and R³ is iso-propyl.

21. The compound according to claim 1, wherein X is in 2, 4 or 6-position, optionally wherein Y and Z independently represent hydrogen, hydroxy, methoxy or methyl.
